(19) United States
(12) Patent Application Publication
(10) Pub. No.: US 2023/0310855 A1
(43) Pub. Date: Oct. 5, 2023

(54) COMPLEX STIMULATION DEVICE

(71) Applicants: Jae Jun SONG, Seoul (KR); NEURIVE Co., Ltd., Gimbae-si, Gyeongsangnam-do (KR); KOREA UNIVERSITY RESEARCH AND BUSINESS, Seoul (KR)

(72) Inventor: Jae Jun SONG, Seoul (KR)

(21) Appl. No.: 18/022,137

(22) PCT Filed: Aug. 18, 2020
(86) PCT No.: PCT/KR2020/010989

§ 371 Date: Feb. 17, 2023

(51) Int. Cl. A61N 1/36; A61N 1/04

(52) U.S. Cl. CPC A61N 1/36036; A61N 1/36014; A61N 1/0476

(57) ABSTRACT

A complex stimulation device includes a contact part composed of multiple layers to come into close contact with an auricle or an external auditory meatus, electric stimulation part inserted and distributed with predetermined gaps inside an outer surface of the contact part, a sound output part inserted through a center of the contact part, and a deforming part formed in the contact part.

### FIELD OF INVENTION

A complex stimulation device is disclosed and, more particularly, a complex stimulation device that is flexibly deformed to effectively electrically stimulate vagus nerves distributed inside the surface of an auricle or an external auditory meatus.

### BACKGROUND ART

In general, tinnitus means a condition of feeling sound stimulation when there is no aural stimulation from the outside. Such tinnitus is classified into objective tinnitus wherein a feeling of internal sounds such as the bloodstream sound or a cramp sound that is transmitted to the ear and subjective tinnitus wherein a person hears meaningless sounds that the patient subjectively makes but other people cannot hear. There are various reasons for tinnitus, but a problem with the nerve system, stress or fatigue, noise, otitis externa, etc. may be the reasons. In order to cure tinnitus, there are medicine treatment and surgical treatment, and it may be possible to cure tinnitus using devices such as a hearing aid and a tinnitus masker.

In relation to tinnitus, a “TINNITUS THERAPY UNIT WHICH USED AN ELECTRICAL STIMULUS AND COOLING WATER” has been disclosed in Korean Patent Application Publication No. 10-2010-0021778 filed on Aug. 18, 2008.

### DISCLOSURE

### Technical Problem

An objective according to an embodiment is to provide a complex stimulation device that effectively electrically stimulates vagus nerves distributed inside the surface of an auricle or an external auditory meatus.

An objective according to an embodiment is to provide a complex stimulation device that can flexibly deform in accordance with the shape of an auricle or an external auditory meatus.

An objective according to an embodiment is to provide a complex stimulation device that uniformly applies electrical stimulation to a wide range of vagus nerves.

An objective according to an embodiment is to provide a complex stimulation device having an electrode of which the length can be adjusted.

An objective according to an embodiment is to provide a complex stimulation device that is inserted and fixed in the entrance of an external auditory meatus.

An objective according to an embodiment is to provide a complex stimulation device having a single standardized shape that can easily come in contact with an auricle or an external auditory meatus that has individually different curves.

Objectives of the present disclosure are not limited to the objectives described above and other objectives will be clearly understood by those skilled in the art from the following description.

### Technical Solution

In order to achieve the objectives, a complex stimulation device according to an embodiment includes: a contact part composed of multiple layers to come into close contact with an auricle or an external auditory meatus;

electric stimulation part inserted and distributed with predetermined gaps inside an outer surface of the contact part; a sound output part inserted through a center of the contact part; and a deforming part formed in the contact part, in which the deforming part is configured to be able to expand and contract when a force is applied to the contact part to bring the contact part into close contact with an auricle or an external auditory meatus, the contact part is flexibly deformed in accordance with a shape of an auricle or an external auditory meatus by expansion and contraction of the deforming part, and the electric stimulation part are brought into close contact with a surface of an auricle or an external auditory meatus by deformation of the contact part and effectively stimulate vagus nerves distributed inside a surface of an auricle or an external auditory meatus.

According to an aspect, the deforming part may be a cavity formed in the contact part and configured to absorb deformation of the contact part.

According to an aspect, the cavity may be disposed around the sound output part and may not interfere with the electric stimulator and the sound output part.

According to an aspect, the contact part may include: a first pad configured to come into contact with an auricle; and

a second pad configured to come into contact with an external auditory meatus, and the second pad may increase in cross-sectional area in a protrusion direction to be inserted and fixed in an entrance of an external auditory meatus.

According to an aspect, the electric stimulation part may include: a first electrode disposed on a top of the first pad to come into contact with a surface of an auricle; and a second electrode disposed on a side of the second pad to come into contact with a surface of an external auditory meatus.

According to an aspect, the first electrode may be inserted in a longitudinal direction of the first pad, and the second electrode may be inserted in a circumferential direction of the second pad.

According to an aspect, a position of each of the electric stimulation part may be adjusted in a longitudinal direction.

According to an aspect, the contact part is made of a flexible insulating material to come into close contact in accordance with a shape of an auricle or an external auditory meatus.

In order to achieve the objectives, a complex stimulation device according to another embodiment includes: a contact part divided into a plurality of sections which composed of multiple layers to come into close contact with an auricle or an external auditory meatus; electric stimulation part inserted and distributed with predetermined gaps inside an outer surface of the contact part; and a sound output part surrounded by the contact part, in which the sections of the contact part are spaced apart from each other around the sound output part, the sections of the contact part are individually flexibly deformed in accordance with a shape of an auricle or an external auditory meatus, and the electric stimulation part are brought into close contact with a surface of an auricle or an external auditory meatus by deformation of the contact part and effectively stimulate vagus nerves distributed inside a surface of an auricle or an external auditory meatus.

According to an aspect, the complex stimulation device may further include connectors positioned between the sections of the contact parts, and the connectors may connect the sections of the contact part to each other.

According to an aspect, the sections of the contact part are arranged with regular intervals around the sound output part.

### Advantageous Effects

According to the complex stimulation device of an embodiment, it is possible to provide a complex stimulation device that can effectively stimulate vagus nerves distributed in an auricle or an external auditory meatus.

According to the complex stimulation device of an embodiment, it is possible to provide a complex stimulation device that flexibly deform in accordance with the shape of an auricle or an external auditory meatus.

According to the complex stimulation device of an embodiment, it is possible to provide a complex stimulation device in which electrodes are uniformly distributed to stimulate vagus nerves in a wide area.

According to the complex stimulation device of an embodiment, it is possible to provide a complex stimulation device in which the length of electrodes can be adjusted.

According to the complex stimulation device of an embodiment, it is possible to provide a complex stimulation device that is inserted and fixed in the entrance of an external auditory meatus.

According to the complex stimulation device of an embodiment, it is possible to provide a complex stimulation device that has a single standardized shape to be able to easily come into close contact with auricles or external auditory meatuses that have individually different curves.

Effects of the complex stimulation device according to an embodiment are not limited to those described above, and other effects not stated above may be clearly understood by those skilled in the art from the following description.

### DESCRIPTION OF DRAWINGS

FIG. 1 is a front view of a complex stimulation device according to an embodiment.

FIG. 2 is a cross-sectional view of the complex stimulation device according to an embodiment.

FIG. 3 shows deformation of the complex stimulation device according to an embodiment.

FIG. 4 is a front view of a complex stimulation device according to another embodiment.

The accompanying drawings of this specification exemplify preferred embodiments and help easy understanding of the present disclosure together with the following detailed description, so the present disclosure should not be construed as being limited to the drawings.

### BEST MODE

Hereafter, embodiments are described in detail with reference to exemplary drawings. It should be noted that when components are given reference numerals in the drawings, the same components are given the same reference numerals even if they are shown in different drawings. Further, in the following description of embodiments, when detailed description of well-known configurations or functions is determined as interfering with understanding of the embodiments, they are not described in detail.

Further, terms “first”, “second”, “A”, “B”, “(a)”, and “(b)” can be used in the following description of the components of embodiments. These terms are provided only for discriminating components from other components and, the essence, sequence, or order of the components are not limited by the terms. When a component is described as being “connected”, “combined”, or “coupled” with another component, it should be understood that the component may be connected or coupled to another component directly or with another component interposing therebetween.

A component included in any one embodiment and a component having the same function are described using the same name in other embodiments. Unless there is contrary statement, description of any one embodiment may be applied to another embodiment and detailed description is omitted within a repeated range.

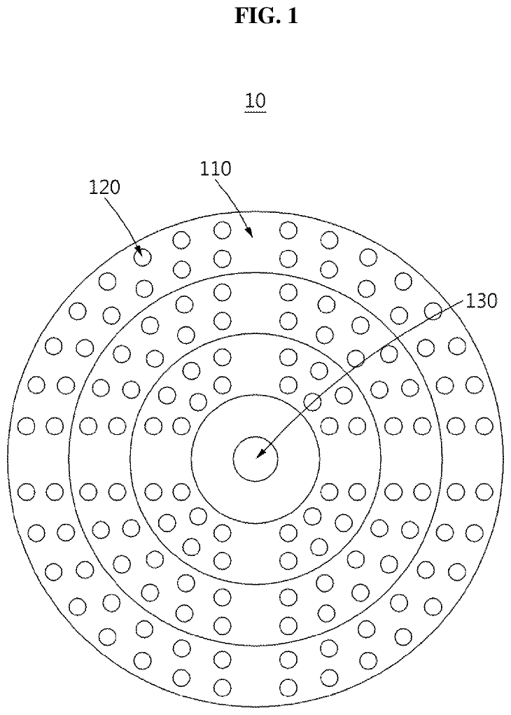

FIG. 1 is a front view of a complex stimulation device according to an embodiment.

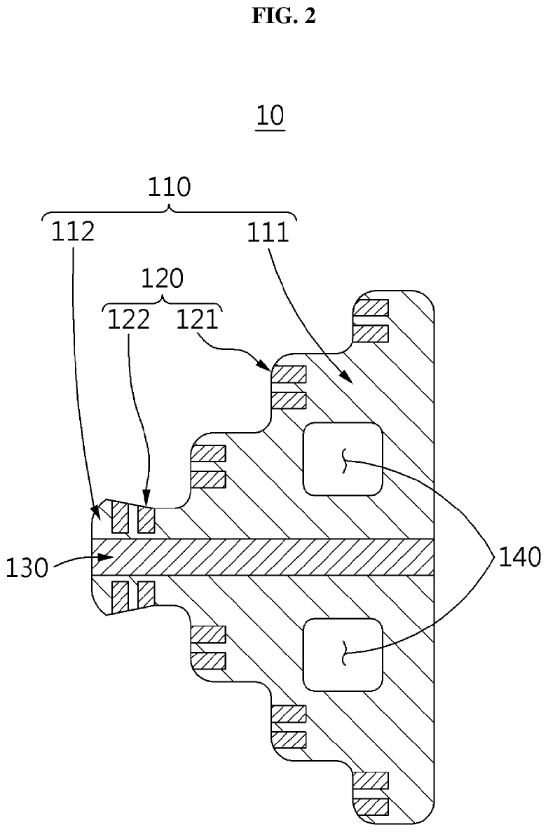

FIG. 2 is a cross-sectional view of the complex stimulation device 10 according to an embodiment.

Referring to FIGS. 1 and 2, the complex stimulation device 10 according to an embodiment may include a contact part 110, electric stimulation part 120, a sound output part 130, and a deforming part 140.

The contact part 110 may be configured to come into contact with a body part to which acoustic and electrical complex stimulation is provided.

Hereafter, the case in which the contact part 110 comes into contact with an auricle or an external auditory meatus to stimulate vagus nerves distributed in an ear is described as an example.

For example, the contact part 110 may be configured to come into close contact with an auricle or an external auditory meatus.

The entire shape of the contact part 110 may be formed similar to the curves of an ear surface. Further, the contact part 110 may be formed in a 3D multi-layer structure to correspond to the curves of an auricle or an external auditory meatus.

The contact part 110 may be made of a flexible material to be easily deformed in accordance with the shape of an auricle or an external auditory meatus and may be made of an insulating material to avoid interference with the electric stimulation part 120.

For example, the contact part 110 may be made of a material such as rubber or silicone, but the material of the contact part 110 is not limited thereto and any material can be used as long as it has flexibility and insulation.

Meanwhile, when all of the parts constituting the contact part 110 are made of a flexible material, portions of the contact part 110 that support the electric stimulation part 120 may also be easily deformed. This may cause a problem that the contact part 110 does not sufficiently support and fix the electric stimulation part 120, depending on the degree of deformation of the contact part 110. Accordingly, the electric stimulation part 120 may be excessively moved or deformed.

Therefore, although it was described above that the contact part 110 may be made of a flexible material such as rubber or silicone to easily deform in accordance with the shape of an auricle or an external auditory meatus, the portions in which the electric stimulation part 120 are inserted and portions of the contact part 110 that surround the electric stimulation part 120 may be partially made of a harder material.

Referring to FIG. 2, the contact part 110 described above may include a first pad 111 and a second pad 112.

In detail, the first pad 111 is curved to come into contact with an auricle and may be formed in a multi-layer structure.

The first pad 111 is symmetrically formed around the sound output part 130 of an auricle in FIGS. 1 and 2, but the first pad 111 is not limited the shape and is not necessarily symmetrically formed. Further, the first pad 111 may be asymmetrically formed around the sound output part 130 and may be composed of various numbers of layers without limitation in the number of layers.

The second pad 112 may protrude further from the top of the first pad 111 toward an external auditory meatus to come into contact with an external auditory meatus.

The second pad 112 is formed such that the cross-sectional area gradually increases in the protrusion direction, so the second pad 112 is inserted in an external auditory meatus with the first pad 111 in close contact with an auricle, whereby the complex stimulation device 10 is easily fixed.

Further, a through-hole (not shown) may be formed in the contact part 110 so that the sound output part 130 is inserted therein.

Referring to FIG. 1, the electric stimulation part 120 may be inserted inside the outer surface of the contact part 110 described above, and electrodes constituting the electric stimulation part 120 may be distributed with regular intervals on the contact part 110.

Referring to FIG. 2 again, the electric stimulation part 120 may include a first electrode 121 and a second electrode 122.

In detail, the first electrode 121 may be disposed on the top of the first pad 111 to come into contact with the surface of an auricle. Further, a plurality of first electrodes 121 may be provided and uniformly distributed on the first pad 111 formed in a multi-layer structure.

In this case, the plurality of first electrodes 121 may be inserted in the first pad 111 in the longitudinal direction of the first pad 111, that is, a direction facing an auricle, thereby being able to come into contact with various portions of the surface of an auricle, respectively.

The second electrode 122 may be disposed on the side of the second pad 112 to come in contact with the surface of an external auditory meatus. Similarly, a plurality of second electrodes 122 may be provided and uniformly distributed on the second pad 112 having a cylindrical shape.

In this case, the plurality of second electrodes 122 may be inserted in the second pad 112 in the circumferential direction of the second pad 112, that is, a direction facing the circumference of an external auditory meatus, thereby being able to uniformly come into contact with the surface of an external auditory meatus.

In addition, the positions of electrode ends of the first electrode 121 and the second electrode 122 may be adjusted in the longitudinal direction of the electrodes such that positions relative to the surfaces of the first pad 111 and the second pad 112 on which the electric stimulation part 120 are disposed are adjusted.

Further, even though the shape of the contact part 110 does not exactly fit the curves and shapes of individuals, it is possible to increase the degree of contact of the electric stimulation part 120 on the surface of an auricle or an external auditory meatus by adjusting the insertion lengths of the first electrode 121 and the second electrode 122.

Accordingly, the arrangement positions and insertion directions of the first electrode 121 and the second electrode 122 enable the electrodes of the complex stimulation device 10 to be easily brought into contact with an ear.

Referring to FIGS. 1 and 2, the sound output part 130 may be disposed in the contact part 110 described above.

The sound output part 130 can stimulate nerves by outputting sound stimulation.

In detail, the sound output part 130 may be inserted through the center of the contact part 110 to pass through both the first pad 111 and the second pad 112, and an output end of the sound output part 130 may be positioned in the same plane as an end of the second pad 112.

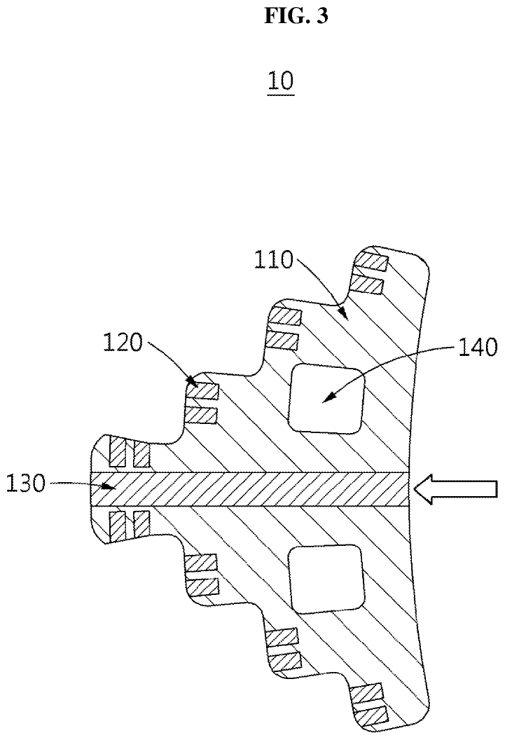

FIG. 3 is a view showing deformation of the complex stimulation device 10 according to an embodiment.

Referring to FIGS. 2 and 3, the deforming part may be formed in the contact part 110 described above.

The deforming part 140 may be an empty cavity to absorb elastic deformation.

In this case, the cavity may be disposed around the sound output part 130 in the contact part 110. Further, the cavity may be formed at a distance that does not generate interference with the electric stimulation part 120 and the sound output part 130.

The deforming part 140 can enable the complex stimulation device 10 to be effectively brought into contact with the surface of the auricle or the external auditory meatus of a user.

Referring to FIG. 3, a user can apply a force that presses the contact part 110 toward an external auditory meatus to put on the complex stimulation device 10 in close contact with an auricle or an external auditory meatus.

In this case, since the deforming part 140 can expand and contract and the shape of the cavity is deformed by the force pressing the contact part 110, the contact part 110 can also be entirely flexibly deformed in accordance with the curves of the auricle or the external auditory meatus of the user.

Accordingly, the electric stimulation part 120 uniformly distributed on the outer surface of the contact part 110 can sufficiently come into contact with the surface of the user's auricle or external auditory meatus.

Further, since the complex stimulation device 10 is fixed inside an external auditory meatus by the shape of the second pad 112, the position of the output end of the sound output part 130 can also be fixed.

Accordingly, since a plurality of electrodes is easily brought into contact with the surface of an auricle or an external auditory meatus due to flexible deformation of the contact part 110 and the deforming part 140 even though users have individually different curves of the auricle or the external auditory meatus, the complex stimulation device 10 according to the present disclosure can effectively stimulate vagus nerves distributed in the ears of users.

Further, it is possible to further increase the treatment effect for patient due to acoustic and electrical complex stimulation.

In addition, the necessity of manufacturing customer-fit devices decreases, so it is possible to manufacture and sell products having a single standardized shape in large quantities and users also can easy access the devices.

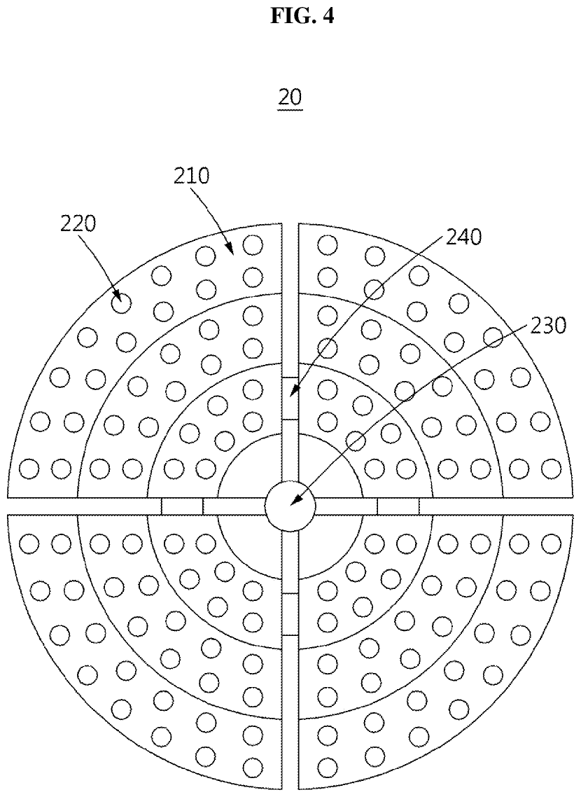

FIG. 4 is a front view of a complex stimulation device according to another embodiment.

Referring to FIG. 4, the complex stimulation device 20 according to another embodiment may include a contact part 210, electric stimulation part 220, a sound output part 230, and connectors 240.

The electric stimulation part 220 and the sound output part 230 of the complex stimulation device 20 shown in FIG. 4 may be the electric stimulation part 120 and the sound output part 130 of the complex stimulation device 10 shown in FIG. 1.

However, the contact part 210 of the complex stimulation device 20 shown in FIG. 4 may have a different configuration from that of the contact part 110 of the complex stimulation device 10 shown in FIG. 1. Further, the complex stimulation device 20 may not have the deforming part 140.

Hereafter, the complex stimulation device 20 is described in detail mainly on the basis of the differences between the complex stimulation device 10 and the complex stimulation device 20.

The contact part 210 of the complex stimulation device according to another embodiment may be divided into a plurality of sections.

Unlike the complex stimulation device 10 in which the sound output part 130 is inserted through the center of the contact part 110, the contact part 210 divided into a plurality of sections in the complex stimulation device 20 may be disposed around the sound output part 230.

The sections of the contact part 210 may be spaced apart from each other around the sound output part 230.

In detail, the contact part 210 may be divided into equal sections and the gaps of the sections may also be the same.

Meanwhile, the entire shape of the contact part 210 may be formed to be similar to the curves of ear surfaces to come into close contact with an auricle or an external auditory meatus. Further, the sections of the contact part 210 may be formed in a 3D multi-layer structure to correspond to the curves of an auricle or an external auditory meatus.

The contact part 210, similarly, may be made of a flexible material that can easily deform and may be made of an insulating material to avoid interference with the electric stimulation part 220.

For example, the contact part 210 may be made of a material such as rubber or silicone, but the material of the contact part 210 is not limited thereto and any material can be used as long as it has flexibility and insulation.

The sections of the contact part 210 each may have first pad and second pad regions, depending on the portions of ear that they are supposed to come into contact with.

Further, the electric stimulation part 220 may be discriminated as a first electrode and a second electrode, depending on the position at which it is disposed on the contact part 210, and the sections of the contact part 210 each may have first electrode and second electrode regions.

The first pad, the second pad, the first electrode, and the second electrode are the same as those described above with reference to FIGS. 1 and 2, so they are not described below.

The complex stimulation device 20 described above may not have the deforming part 140 in the contact part 210. However, the complex stimulation device 20 may include connectors 240 connecting the sections of the contact part 210 to each other.

The connectors 240 are positioned between the sections of the contact part 210 and both ends of each of the connectors 240 may be attached to different sections, respectively.

Further, the connectors 240 may be made of a flexible material having elasticity. Accordingly, even though an excessive force is applied to the contact part 210, the sections of the contact part 210 can be individually moved and deformed within a predetermined range by elasticity of the connectors 240.

As a result, the sections of the contact part 210 and the connectors 240 connecting the sections in the complex stimulation device 20 according to another embodiment can effectively bring the complex stimulation device 20 into contact with the surface of the auricle or the external auditory meatus of a user.

In detail, a user can apply a force that presses the entire contact part 210 toward an external auditory meatus to put on the complex stimulation device 20 in close contact with an auricle or an external auditory meatus.

In this case, the sections of the contact part 210 can be individually flexibly deformed in accordance with the curves of the user's auricle or external auditory meatus by the force pressing the contact part 210.

Further, the sections can be elastically deformed with respect to each other by the connectors 240.

Accordingly, the electric stimulation part 220 uniformly distributed on the outer surfaces of the sections of the contact part 210 can sufficiently come into contact with the surface of the user's auricle or external auditory meatus.

Further, since the complex stimulation device 20 is fixed inside an external auditory meatus by the shape of the second pad 112, the position of the output end of the sound output part 230 can also be fixed.

Accordingly, even though users have individually different curves of an auricle or an external auditory meatus, the complex stimulation device 20 according to an embodiment can be more flexibly deformed by the sections of the contact part 210 that can be individually deformed and the connectors 240 connecting the sections.

Further, a plurality of electrodes can be easily brought into contact with surfaces in accordance with curves of an auricle or an external auditory meatus, so it is possible to effectively stimulate the vagus nerves distributed in a wide range of an ear of a user.

Further, it is possible to further increase the treatment effect for patient due to acoustic and electrical complex stimulation.

In addition, the necessity of manufacturing customer-fit devices decreases, so it is possible to manufacture and sell products having a single standardized shape in large quantities and users also can easy access the devices.

Hereinabove, although the present invention is described by specific matters such as concrete components, and the like, embodiments, and drawings, they are provided only for assisting in the entire understanding of the present invention. Therefore, the present invention is not limited to the embodiments. Various modifications and changes may be made by those skilled in the art to which the present invention pertains from this description. For example, the described technologies may be performed in order different from the described method, and/or even if components such as the described structure and device are combined or associated in different ways from the description or replaced by other components or equivalents, appropriate results can be accomplished. Therefore, the spirit of the present invention should not be limited to the above-described embodiments, and the following claims as well as all modified equally or equivalently to the claims are intended to fall within the scope and spirit of the invention.

1. A complex stimulation device comprising:
a contact part composed of multiple layers to come into close contact with an auricle or an external auditory meatus;
electric stimulation part inserted and distributed with predetermined gaps inside an outer surface of the contact part;
a sound output part inserted through a center of the contact part; and
a deforming part formed in the contact part,
wherein the deforming part is configured to be able to expand and contract when a force is applied to the contact part to bring the contact part into close contact with an auricle or an external auditory meatus,
the contact part is flexibly deformed in accordance with a shape of an auricle or an external auditory meatus by expansion and contraction of the deforming part, and
the electric stimulation part are brought into close contact with a surface of an auricle or an external auditory meatus by deformation of the contact part and effectively stimulate vagus nerves distributed inside a surface of an auricle or an external auditory meatus.

2. The complex stimulation device of claim 1, wherein the deforming part is a cavity formed in the contact part and configured to absorb deformation of the contact part.

3. The complex stimulation device of claim 2, wherein the cavity is disposed around the sound output part and does not interfere with the electric stimulator and the sound output part.

4. The complex stimulation device of claim 1, wherein the contact part includes:
a first pad configured to come into contact with an auricle; and
a second pad configured to come into contact with an external auditory meatus,
wherein the second pad increases in cross-sectional area in a protrusion direction to be inserted and fixed in an entrance of an external auditory meatus.

5. The complex stimulation device of claim 4, wherein the electric stimulation part include:
a first electrode disposed on a top of the first pad to come into contact with a surface of an auricle; and
a second electrode disposed on a side of the second pad to come into contact with a surface of an external auditory meatus.

6. The complex stimulation device of claim 5, wherein the first electrode is inserted in a longitudinal direction of the first pad, and
the second electrode is inserted in a circumferential direction of the second pad.

7. The complex stimulation device of claim 1, wherein a position of each of the electric stimulation part can be adjusted in a longitudinal direction.

8. The complex stimulation device of claim 1, wherein the contact part is made of a flexible insulating material to come into close contact in accordance with a shape of an auricle or an external auditory meatus.

9. A complex stimulation device comprising:
a contact part divided into a plurality of sections and composed of multiple layers to come into close contact with an auricle or an external auditory meatus;
electric stimulation part inserted and distributed with predetermined gaps inside an outer surface of the contact part; and
a sound output part surrounded by the contact part,
wherein the sections of the contact part are spaced apart from each other around the sound output part,
the sections of the contact part are individually flexibly deformed in accordance with a shape of an auricle or an external auditory meatus, and
the electric stimulation part are brought into close contact with a surface of an auricle or an external auditory meatus by deformation of the contact part and effectively stimulate vagus nerves distributed inside a surface of an auricle or an external auditory meatus.

10. The complex stimulation device of claim 9, further comprising connectors positioned between the sections of the contact parts,
wherein the connectors connect the sections of the contact part to each other.

11. The complex stimulation device of claim 9, wherein the sections of the contact part are arranged with regular intervals around the sound output part.
